(19) United States
(12) Patent Application Publication
(10) Pub. No.: US 2021/0090734 A1
(43) Pub. Date: Mar. 25, 2021

(54) SYSTEM, DEVICE AND METHOD FOR DETECTION OF VALVULAR HEART DISORDERS

(71) Applicants: Kaushik Kunal SINGH, PLEASANTON, CA (US); Sachin Saagar SINGH, PLEASANTON, CA (US)

(72) Inventors: Kaushik Kunal SINGH, PLEASANTON, CA (US); Sachin Saagar SINGH, PLEASANTON, CA (US)

(21) Appl. No.: 16/576,797

(22) Filed: Sep. 20, 2019

(51) Int. Cl. G16H 50/20; G06N 3/08

(52) U.S. Cl. CPC G16H 50/20; G06N 3/08

(57) ABSTRACT

The present disclosure provides a system, device and method for detection of valvular heart disorders. The system includes: a recording unit configured to record set of heart sounds and store the set of heart sounds in a database operatively coupled to the recording unit; and a control unit configured to: segment the set of heart sounds into a plurality of slices, each having one or more audio slices; convert the audio slices into corresponding spectrograms; obtain a feature vector corresponding to the spectrograms; compare the obtained feature vector with a predetermined set of feature vectors stored in the database; and classify each of the spectrograms into either a normal spectrogram or an abnormal spectrogram, based on the comparison of the obtained feature vector with the predetermined set of feature vectors, to obtain classification scores associated with the spectrograms.

### TECHNICAL FIELD

The present disclosure relates to the field of systems and methods for detecting diseases. More particularly, the present disclosure relates to a system and method for detection of valvular heart disorders such as murmurs etc.

### BACKGROUND

Background description includes information that may be useful in understanding the present invention. It is not an admission that any of the information provided herein is prior art or relevant to the presently claimed invention, or that any publication specifically or implicitly referenced is prior art.

According to world health organization (WHO), heart diseases are one of prime reasons for causing deaths globally, with three-quarters occurring in developing countries. Rural mortality rates have surpassed those of urban areas as 75% of rural primary care centres are handled by unqualified medical practitioners due to shortage of trained doctors. A 2017 study of Medical Council of India's historical data shows that there were only 4.8 practicing doctors per 10,000 of population. Hence, a majority of rural population turns to informal healthcare workers who provide approximately 75 percent of primary care, but have no formal medical training, hence worsening the cardiac risks. Some of the most common forms of heart disorders are valvular resulting in murmurs that claim millions of lives every year.

Auscultation is typically used as a diagnosis tool in medicine, in particular for the diagnosis of cardiovascular diseases. Auscultation relies on correctly determining which of the primary heart sounds correspond to the systolic phase of the heart and which sounds correspond to the diastolic phase of the heart. Learning auscultation may be difficult. The skill relies on detecting a correct sequence of brief events that occur close in time, a skill that is often difficult for human listeners. This is made more difficult when the systolic and diastolic intervals become equal, and which typically occurs at elevated heartrates. As mentioned-above, auscultation, a process of listening to internal sounds of the body, has historically been performed with acoustic stethoscopes. Auscultation of heart sound recordings has been shown to be valuable for the detection of disease and pathologies. Many different forms of such a device have existed; most notably those are included with a two-sided chest piece linked with branched hollow tubing to two separate earpieces. Such devices use a diaphragm to transmit high frequency sounds to a doctor's ears, and a bell to transmit the low frequency sounds. However, the common acoustic stethoscope lacks the ability to digitize sounds for further medical use.

An auscultation training is available, for example, recordings of heart sounds, i.e. phonocardiograms (PCGs), that emphasize various pathological conditions exist. Many of these recordings are processed to emphasize particular features, such as enhancing a mid-systolic click to better distinguish the features of mitral valve prolapse. The recordings are also typically processed to reduce background noise commonly found in clinical practice as a physician new to clinical practice may have difficulty in recognizing the distinguishing sounds included with background noise in a clinical setting. Alternatively, training systems that use simulated heart sounds exist. Some of the simulated sounds, however, may seem unnatural to a trained physician or, as simulated, may not be physiologically feasible. Furthermore, teaching auscultation typically relies on memorization of heart sound patterns related to a particular pathophysiological condition.

Signals obtained by means of a transducer are phonocardiographic representations of sounds traditionally listened, by means of the stethoscope, by a user (such as doctors, medical practitioners etc.). Training in auscultation takes a long time and requires an aptitude for recognising and classifying aural cues, frequently in a noisy environment. 20-30 different conditions may need to be differentiated, and within each, the severity evaluated. Furthermore, there may be combinations among these. These factors contribute to explaining why not all physicians perform equally well when diagnosing heart conditions, and why it may be time-consuming.

Additionally, diagnostic instructional manuals rely on subjective descriptions of heart sounds, such as “musical” or “blowing” sounding murmurs, which require practice to appreciate. Furthermore, the practice and teaching of the clinical skill of auscultation of the heart has declined among physicians, partly due to a reliance on echocardiography testing. Recent studies have concluded that physicians may reliably identify only a small number of standard heart sounds and murmurs. Consequently, serious heart murmurs may go undetected by physicians. Further, some of medical practitioners may fail to detect or differentiate between abnormal and normal heart sounds to make an assessment for referrals to more advanced and expensive testing such as electrocardiogram (ECG) etc.

Therefore, there is a need in the art to provide a means for early detection of heart disorders and/or diseases in various patients with enhanced accuracy. Further, there is a need to help untrained medical practitioners and health workers at remote places (such as villages, forest areas or any other rural areas) in detecting various heart related disorders efficiently.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities or dimensions of items, and so forth, used to describe and claim certain embodiments of the invention are to be understood as being modified in some instances by the term “about.” Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Groupings of alternative elements or embodiments of the invention disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all groups used in the appended claims.

### OBJECTS OF THE PRESENT DISCLOSURE

Some of the objects of the present disclosure, which at least one embodiment herein satisfies are as listed herein below.

It is an object of the present disclosure to provide a system, device and method for early detection or screening of heart related disorders or abnormalities such as murmurs etc. in a patient.

It is another object of the present disclosure to provide a simple and cost effective system, device and method for early detection of valvular heart disorders in a patient.

It is another object of the present disclosure to provide a reliable, efficient and accurate system, device and method for early detection of valvular heart disorders in a patient.

It is another object of the present disclosure to provide an automated stethoscope based cardiac auscultation to detect abnormal heart sound to save patients from heart related diseases.

### SUMMARY

The present disclosure relates to the field of systems and methods for detecting diseases. More particularly, the present disclosure relates to a system and method for detection of valvular heart disorders such as murmurs etc.

This summary is provided to introduce simplified concepts of a system for time bound availability check of an entity, which are further described below in the detailed description. This summary is not intended to identify key or essential features of the claimed subject matter, nor is it intended for use in determining/limiting the scope of the claimed subject matter.

An aspect of the present disclosure pertains to a system for early detection of valvular heart disorders in a patient. The system can include: a recording unit that can be configured to record a set of heart sounds of the patient and store the set of heart sounds in a database operatively coupled to the recording unit; and a control unit having processors and a memory that can be operatively coupled to the processors. The memory storing instructions can be executable by the processors to enable the control unit to: segment the set of heart sounds into a plurality of slices, each of a predetermined length, and each of the plurality of slices can include at least one audio slice; convert the at least one audio slice into corresponding spectrograms; obtain a feature vector corresponding to the spectrograms; compare the obtained feature vector with a predetermined set of feature vectors that can be stored in the database; and classify each of the spectrograms into any or a combination of a normal spectrogram and an abnormal spectrogram, based on the comparison of the obtained feature vector with the predetermined set of feature vectors, to obtain classification scores associated with the spectrograms.

In an aspect, the spectrograms are time-based spectrograms.

In an aspect, the control unit can be configured to classify, using a deep convolutional neural network (CNN) trained model, each of the spectrograms into any or a combination of the normal spectrogram and the abnormal spectrogram.

In an aspect, the system can be configured to: compute any or a combination of a mean and standard deviation of the classification scores to remove any deviation, if present, in the classification scores; and store, in the database, an audio slice corresponding to an obtained higher classification score. The purpose of storing the higher classification score is to enable retraining of the CNN model.

In an aspect, the CNN trained model can be configured to, based on any or a combination of the classification scores, the mean and the standard deviation of the classification scores, detect at least one of heart sound patterns and valvular heart disorders associated with the patient.

In an aspect, the valvular heart disorders can include at least one of a murmur and an arrhythmia, and the murmur can be at least one of a systolic murmur, late systolic murmur, holosystolic murmur and early diastolic murmur.

Another aspect of the present disclosure pertains to a method for early detection of valvular heart disorders in a patient. The method can include steps of: recording, at a recording unit, a set of heart sounds of the patient and storing the set of heart sounds in a database; segmenting, by a control unit having processors and a memory, the set of heart sounds into a plurality of slices, each of a predetermined length, and the plurality of slices having at least one audio slice; converting, by the control unit, the at least one audio slice into corresponding spectrograms; obtaining, by the control unit, a feature vector corresponding to the spectrograms; comparing, by the control unit, the obtained feature vector with a predetermined set of feature vectors stored in the database; and classifying each of the spectrograms into any or a combination of a normal spectrogram and an abnormal spectrogram, based on said comparison of the obtained feature vector with the predetermined set of feature vectors, to obtain classification scores associated with the spectrograms.

In an aspect, at the step of classifying each of the spectrograms, the control unit can be configured to classify, using a deep convolutional neural network (CNN) trained model, each of the spectrograms into any or a combination of the normal spectrogram and the abnormal spectrogram.

In an aspect, the method can include steps of: computing, at the processors, any or a combination of a mean and standard deviation of the classification scores to remove any deviation, if present, in the classification scores; and storing, in the database, an audio slice corresponding to an obtained higher classification score.

Another aspect of the present disclosure pertains to a device early detection of valvular heart disorders in a patient. The device can include processors and a memory operatively coupled with the processors. The memory can store instructions executable by the processors to enable the device to: receive, using a transceiving unit, a recorded set of heart sounds of the patient from a recording unit that can be operatively coupled to the device; segment, using a segmentation unit, the received set of heart sounds into a plurality of slices, each of a predetermined length, and the plurality of slices can include at least one audio slice; convert, using a converting unit, the at least one audio slice into corresponding spectrograms; obtain, at the processors, a feature vector corresponding to the spectrograms; compare, at the processors, the obtained feature vector with a predetermined set of feature vectors stored in a database operatively coupled to the device; and classify, at the processors, each of the spectrograms into any or a combination of a normal spectrogram and an abnormal spectrogram, based on said comparison of the obtained feature vector with the predetermined set of feature vectors, to obtain classification scores associated with the spectrograms.

Various objects, features, aspects and advantages of the inventive subject matter will become more apparent from the following detailed description of preferred embodiments, along with the accompanying drawing figures in which like numerals represent like components

### BRIEF DESCRIPTION OF THE DRAWINGS

The diagrams are for illustration only, which thus is not a limitation of the present disclosure, and wherein:

FIG. 1 illustrates an exemplary block diagram representation of a system for early detection of valvular heart disorders in a patient, in accordance with an embodiment of the present disclosure.

FIG. 2 illustrates an exemplary flow diagram representation of method for early detection of valvular heart disorders in a patient, in accordance with an embodiment of the present disclosure.

FIG. 3 illustrates an exemplary representation of heartbeat collected in real-life and visualized with audacity, in accordance with an embodiment of the present disclosure.

FIG. 4 illustrates an exemplary flow diagram representation of an algorithm for automated screening, in accordance with an embodiment of the present disclosure.

FIG. 5 illustrates an exemplary end-to-end system architecture for diagnostic tool, in accordance with an embodiment of the present disclosure.

FIG. 6 illustrates an exemplary representation of digital stethoscope, in accordance with an embodiment of the present disclosure.

FIG. 7 illustrates an exemplary representation of mobile device in connection with digital stethoscope, in accordance with an embodiment of the present disclosure.

FIG. 8 illustrates an exemplary representation of spectrogram of real heart sample collected with stethoscope, in accordance with an embodiment of the present disclosure.

FIG. 9A illustrates an exemplary block diagram representation of device for early detection of valvular heart disorders in a patient, in accordance with an embodiment of the present disclosure.

FIG. 9B illustrates an exemplary schematic representation of the device of FIG. 9A with recording unit and database, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

In the following description, numerous specific details are set forth in order to provide a thorough understanding of embodiments of the present invention. It will be apparent to one skilled in the art that embodiments of the present invention can be practiced without some of these specific details.

Embodiments of the present invention include various steps, which will be described below. The steps can be performed by hardware components or can be embodied in machine-executable instructions, which can be used to cause a general-purpose or special-purpose processor programmed with the instructions to perform the steps. Alternatively, steps can be performed by a combination of hardware, software, and firmware and/or by human operators.

The present disclosure relates to the field of systems and methods for detecting diseases. More particularly, the present disclosure relates to a system and method for detection of valvular heart disorders such as murmurs etc.

An aspect of the present disclosure pertains to a system for early detection of valvular heart disorders in a patient. The system can include: a recording unit that can be configured to record a set of heart sounds of the patient and store the set of heart sounds in a database operatively coupled to the recording unit; and a control unit having processors and a memory that can be operatively coupled to the processors. The memory is storing instructions executable by the processors to enable the control unit to: segment the set of heart sounds into a plurality of slices, each of a predetermined length, and each of the plurality of slices can include at least one audio slice; convert the at least one audio slice into corresponding spectrograms; obtain a feature vector corresponding to the spectrograms; compare the obtained feature vector with a predetermined set of feature vectors that can be stored in the database; and classify each of the spectrograms into any or a combination of a normal spectrogram and an abnormal spectrogram, based on the comparison of the obtained feature vector with the predetermined set of feature vectors, to obtain classification scores associated with the spectrograms.

In an aspect, the spectrograms are time-based spectrograms.

In an aspect, the control unit can be configured to classify, using a deep convolutional neural network (CNN) trained model, each of the spectrograms into any or a combination of the normal spectrogram and the abnormal spectrogram. A transfer learning can be implemented to retrain the trained convolutional neural network in order to classify spectrogram images as either normal or abnormal. Further, the transfer learning can be used to update or modulate the trained CNN model quickly by replacing final layers of the CNN model with the help of a small set of training images.

In an aspect, the system can be configured to: compute any or a combination of a mean and standard deviation of the classification scores to remove any deviation, if present, in the classification scores; and store, in the database, an audio slice corresponding to an obtained higher classification score. The purpose of storing the higher classification score is to enable retraining of the CNN model.

In an aspect, the CNN trained model can be configured to, based on any or a combination of the classification scores, the mean and the standard deviation of the classification scores, detect at least one of heart sound patterns and valvular heart disorders associated with the patient.

In an aspect, the valvular heart disorders can include at least one of a murmur and an arrhythmia, and the murmur can be at least one of a systolic murmur, late systolic murmur, holosystolic murmur and early diastolic murmur.

Another aspect of the present disclosure pertains to a method for early detection of valvular heart disorders in a patient. The method can include steps of: recording, at a recording unit, a set of heart sounds of the patient and storing the set of heart sounds in a database; segmenting, by a control unit having processors and a memory, the set of heart sounds into a plurality of slices, each of a predetermined length, the plurality of slices comprises at least one audio slice; converting, by the control unit, the at least one audio slice into corresponding spectrograms; obtaining, by the control unit, a feature vector corresponding to the spectrograms; comparing, by the control unit, the obtained feature vector with a predetermined set of feature vectors stored in the database; and classifying each of the spectrograms into any or a combination of a normal spectrogram and an abnormal spectrogram, based on said comparison of the obtained feature vector with the predetermined set of feature vectors, to obtain classification scores associated with the spectrograms.

In an aspect, at the step of classifying each of the spectrograms, the control unit can be configured to classify, using a deep convolutional neural network (CNN) trained model, each of the spectrograms into any or a combination of the normal spectrogram and the abnormal spectrogram.

In an aspect, the method can include steps of: computing, at the processors, any or a combination of a mean and standard deviation of the classification scores to remove any deviation, if present, in the classification scores; and storing, in the database, an audio slice corresponding to an obtained higher classification score.

Another aspect of the present disclosure pertains to a device early detection of valvular heart disorders in a patient. The device can include processors and a memory operatively coupled with the processors. The memory can store instructions executable by the processors to enable the device to: receive, using a transceiving unit, a recorded set of heart sounds of the patient from a recording unit that can be operatively coupled to the device; segment, using a segmentation unit, the received set of heart sounds into a plurality of slices, each of a predetermined length, and the plurality of slices can include at least one audio slice; convert, using a converting unit, the at least one audio slice into corresponding spectrograms; obtain, at the processors, a feature vector corresponding to the spectrograms; compare, at the processors, the obtained feature vector with a predetermined set of feature vectors stored in a database operatively coupled to the device; and classify, at the processors, each of the spectrograms into any or a combination of a normal spectrogram and an abnormal spectrogram, based on said comparison of the obtained feature vector with the predetermined set of feature vectors, to obtain classification scores associated with the spectrograms.

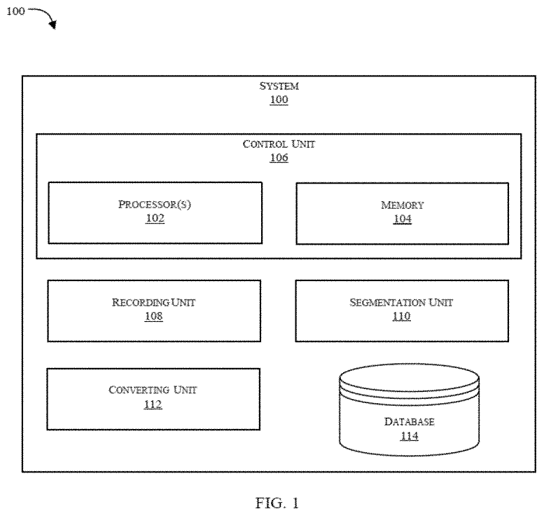

FIG. 1 illustrates an exemplary block diagram representation of a system for early detection of valvular heart disorders in a patient, in accordance with an embodiment of the present disclosure.

According to an embodiment, the system 100 can include one or more processor(s) 102. The one or more processor(s) 102 can be implemented as one or more microprocessors, microcomputers, microcontrollers, digital signal processors, central processing units, logic circuitries, and/or any devices that manipulate data based on operational instructions. Among other capabilities, the one or more processor(s) 102 are configured to fetch and execute computer-readable instructions stored in a memory 104 of the system 100. The memory 104 can store one or more computer-readable instructions or routines, which can be fetched and executed to create or share the data units over a network service. The memory 104 can include any non-transitory storage device including, for example, volatile memory such as RAM, or non-volatile memory such as EPROM, flash memory, and the like.

Various components /units of the proposed system 100 can be implemented as a combination of hardware and programming (for example, programmable instructions) to implement their one or more functionalities as elaborated further themselves or using processors 102. In examples described herein, such combinations of hardware and programming can be implemented in several different ways. For example, the programming for the units can be processor executable instructions stored on a non-transitory machine-readable storage medium and the hardware for units can include a processing resource (for example, one or more processors), to execute such instructions. In the present examples, the machine-readable storage medium can store instructions that, when executed by the processing resource, implements the various units. In such examples, the system 100 can include the machine-readable storage medium storing the instructions and the processing resource to execute the instructions, or the machine-readable storage medium can be separate but accessible to the system 100 and the processing resource. In other examples, the units can be implemented by electronic circuitry. A database 114 can include data that is either stored or generated as a result of functionalities implemented by any of the other components /units of the proposed system 100.

In an embodiment, the system 100 for early detection of valvular heart disorders is disclosed. The system 100 can include: a recording unit 108; and a control unit 106 having processors 102 and memory 104 that can be operatively coupled with the processors 102.

### Recording Unit 108

The recording unit 108 can be configured to record a set of heart sounds and store the set of heart sounds in the database 114 that can be operatively coupled to the recording unit 108.

In an exemplary embodiment, the recording unit 108 can include digital stethoscope to record heart sounds of the patient. The digital stethoscope can be built using an analog stethoscope head, an 8 mm lapel mike (Omni-directional electrical condenser with signal-to-noise ratio of 50 dB to 100 dB, sensitivity of around −30 dB, frequency range of 65 Hz-18 KHz), and a foam insulator with vinyl tube to provide a sound insulated connection. To help record low amplitude and lower frequency band heart sound, the sensitivity can be maximized; a low pass filter can be used, and a 32-bit floating-point sampling at rate of 44 KHz can be used for recording.

The heart pulsation results in flow and circulation of the blood. There will be changes in tissue form and fluid mechanics during the pulsation period. Sounds emitted due to these changes can be heard by using the stethoscope. These sounds are called heart tones. Heart murmurs are caused by turbulent flow of the blood. They can be divided into the systolic period, the diastolic period and the sustaining period according to the occurrence time. They can also be divided into the aortic valve, the pulmonary valve, the tricuspid valve and the mitral valve according to the diagnosis positions. A valve is like a door for controlling the blood to flow in a certain direction. For instance, the aortic valve is located between the left ventricle and the main artery, and controls the blood supply of the whole human body. For a patient with a narrow aortic valve, there will be a pressure difference between the left ventricle and the main artery when the heart contracts. The narrower the aortic valve, the larger the pressure difference. A doctor can thus find the heart murmur of the systolic period.

The heart tones can be divided into a first heart tone, a second heart tone, a third tone and a fourth heart tone. The first heart tone occurs at the initial stage when the heart contracts, and includes two components caused by the closure of the mitral valve and the tricuspid valve. The second heart tone occurs at the last phase when the heart contracts, and includes two components caused by the aortic valve and the pulmonary valve. The third heart tone occurs at the initial stage when the heart expands. The fourth heart tone occurs at the last phase when the heart expands. The first and second heart tones are sounds generated when the valves close and are thus easy to observe. The third and fourth heart tones are less apparent and thus difficult to observe. Abnormal sounds, sounds other than these four heart tones, are viewed as heart murmurs. These heart murmurs represent symptoms of heart diseases including valve stenosis, valve regurgitation, valve cracks, or other defects in structure.

The recording unit 108 can include heart tone microphones that can be used for digital processing of the heart tones recorded by one or more heart tone microphones, and for operation and comparison of system transfer functions. The heart tone signals of the heart valves can be obtained by subtracting a heart tone measured at the pulmonary valve from the heart tone measured at the aortic valve, subtracting the heart tone measured at the aortic valve from the heart tone measured at the pulmonary valve, subtracting the heart tone measured at the tricuspid valve from the heart tone measured at the mitral valve, and subtracting the heart tone measured at the mitral valve from the heart tone measured at the tricuspid valve. The heart tone signals can also be obtained in other ways of operations. To record heart sounds, other types of sound-detecting sensors or microphones can also be used, such as pressure sensors or vibration sensors configured to respond to sounds made by the heart.

In an exemplary embodiment, the recording unit 108 includes a plurality (two or more) of sound-detecting sensors. The plurality of sensed heart sound signals from the plurality of sensors can be individually transmitted to an external system for display as individual traces, can be combined (e.g., averaged) by external system before being displayed as a single trace, or can be combined by control unit 106 before being transmitted to external system as a single heart sound signal. These sensors can include different types of sensors, sensors that are located in different locations, or sensors that generate sensed signals, which receive different forms of signal processing.

In an exemplary embodiment, the recording unit 108 can include an accelerometer that can be configured to generate sensed signals representative of two distinct physical parameters: (1) the level of activity of the patient; and (2) the heart sounds generated by heart. Accordingly, analog pre-processing circuit of the recording unit 108 can be configured to pre-process the sensed signals from accelerometer in a manner that conforms to the signal characteristics of both of these physical parameters. For example, if the frequencies of interest for measuring the patient's level of activity are below 10 Hz, while the frequencies of interest for detecting heart sounds are between 0.05 Hz and 50 Hz, then analog pre-processing circuit can include a low-pass filter having a cut-off frequency of 50 Hz. The control unit 106 can then perform additional filtering in software using, for example, a low-pass filter with a cut-off frequency of 10 Hz to detect the level of activity of the patient, and a band-pass filter with cut-off frequencies of 0.05 Hz and 50 Hz to detect the heart sounds, although these signal processing functions can also be performed by external system. Along with filtering, analog pre-processing circuit can perform other processing functions including automatic gain control (AGC) functions.

Referring to FIG. 1, the control unit 106 can be operatively coupled to a segmentation unit 110. The segmentation unit 110 can be configured to control the processors 102 to segment the set of heart sounds into a plurality of slices with each slice of a predetermined length. Each of the plurality of slices can include at least one audio slice. The set of heart sounds can be subdivided into multiple (e.g., overlapping or non-overlapping) time slices.

In some exemplary embodiments, each time slice can represent a twenty (20) millisecond portion of the heart sound. According to various exemplary embodiments, the time slices can have a uniform duration that ranges from ten (10) milliseconds to thirty (30) milliseconds.

In an exemplary embodiment, a slice can be composed of a string of consecutive macroblocks, which is commonly built from a 2 by 2 matrix of blocks, and it allows error resilience in case of data corruption. Due to the existence of a slice in an error resilient environment, a partial picture can be constructed instead of the whole picture being corrupted. If a bit stream contains an error, then a decoder can skip to the start of the next slice. Having more slices in the bit stream allows better error hiding, but it can use space that could otherwise be used to improve audio quality. The slice is composed of macroblocks traditionally running from left to right and top to bottom where all macroblocks in the I-pictures are transmitted. In P and B-pictures, typically, some macroblocks of a slice are transmitted and some are not, that is, they are skipped. However, the first and last macroblock of a slice can be transmitted. In addition, the slices may not overlap.

In an exemplary embodiment, a heart sound segment can have an analog or digital representation. In some embodiments, a heart sound segment can be represented by a sequence of digitally sampled sound samples. A sound segment can represent a time slice of any other body organs; or a time slice of many studio-mixed sounds of body organs; or any other type of sounds. During playback, many heart sound segments can be combined together in alternative ways to form each channel. In some embodiments, a heart sound segment can also be defined by a sequence of MIDI-like commands that control one or more instruments that can generate the heart sound segment. In some embodiments, during playback, each MIDI-like segment (command sequence) can be converted to a digitally sampled sound segment before being combined with other sound segments. In some embodiments, some sound segments can initiate a variable selection of alternative sound segments during playback. MIDI-like segments can have the same initiation capabilities as other sound segments. In some embodiments, pointers/parameters can be used to identify the location/beginning of a heart sound segment and the segment's length/ending.

In an exemplary embodiment, a first clip (i.e. typically noisy) and a last clip (as its time period may not be same) of each slice can be discarded.

### Converting Unit 112

In an embodiment, the control unit 106 can be operatively coupled to the converting unit 112. The converting unit 112 can control the processors 102 to convert the at least one audio slice into corresponding spectrograms.

In an exemplary embodiment, each heart sound can be converted from a typical audio format (e.g., mp3, way, etc.) to a mel-frequency spectrogram with tilt and amplitude normalization over a pre-selected frequency range (400 Hz to 4 kHz). For computational efficiency, the input audio can be low-pass filtered to about 5/4 of the top of the selected frequency range and then down sampled accordingly. For example, using 4 kHz as the top of our frequency range of interest and using 44.1 kHz as the input audio sampling rate, the input audio can be low-pass filtered using a simple finite impulse response (FIR) filter with an approximate frequency cut between 5 and 5.5 kHz and then subsampled to a 11.025 kHz sampling rate. To minimize volume-change effects, the audio sample energy can be normalized using the local average energy, taken over a tapered, centred 10-second window. To minimize aperture artifacts, the average energy can also be computed using a tapered Hamming window.

A spectrogram “slice rate” of 100 Hz (that is, a slice step size of 10 ms) can be used. For the slices, audio data can be taken, a tapered window (to avoid discontinuity artifacts in the output) applied, and then an appropriately sized Fourier transform can be applied. The Fourier magnitudes are “de-tilted” using a single-pole filter to reduce the effects of low-frequency bias and then “binned” (averaged) into B frequency samples at mel-scale frequency spacing (e.g., B=32).

Referring to FIG. 1, in an embodiment, the control unit 106 can be configured to obtain at least one feature vector corresponding to the spectrograms.

In an embodiment, the control unit 106 can be configured to compare the obtained at least one feature vector with a predetermined set of feature vectors that are stored in the database 114.

In an embodiment, the control unit 106 can be configured to classify each of the spectrograms into any or a combination of a normal spectrogram and an abnormal spectrogram, based on said comparison of the obtained feature vector with the predetermined set of feature vectors, in order to obtain classification scores associated with the spectrograms. In an embodiment, the spectrograms can be time-based spectrograms.

In an embodiment, the control unit 106 can be configured to classify, using a deep convolutional neural network (CNN) trained model, each of the spectrograms into any or a combination of the normal spectrogram and the abnormal spectrogram. This can help in detecting valvular heart disorders of the patient efficiently and accurately in a reliable manner. The implementation of CNN is cost-effective as well as compared other existing processing techniques.

In an exemplary embodiment, Convolutional neural networks are a type of feed-forward artificial neural networks. Convolutional neural networks can include collections of neurons that each have a receptive field and that collectively tile an input space. Convolutional neural networks (CNNs) have numerous applications. In particular, CNNs have broadly been used in the area of pattern recognition and classification. Deep learning architectures, such as deep belief networks and deep convolutional networks, are layered neural networks architectures in which the output of a first layer of neurons becomes an input to a second layer of neurons, the output of a second layer of neurons becomes an input to a third layer of neurons, and so on. Deep neural networks can be trained to recognize a hierarchy of features and so they have increasingly been used in object recognition applications. Like convolutional neural networks, computation in these deep learning architectures can be distributed over a population of processing nodes, which can be configured in one or more computational chains. These multi-layered architectures can be trained one layer at a time and can be fine-tuned using back propagation.

In an embodiment, the system 100 can collect heart sound samples and plot their spectrograms that are visual representations of a spectrum of frequencies of sound as a function of time. This collected data can be utilized for training the CNN trained model on the spectrogram dataset generated from heart sound training dataset. Once the trained CNN model is ready for real-life testing of trained model on real heart sounds, heart sounds of patient can be recorded using low cost digital stethoscope that can be in a communication with mobile application installed on any remote mobile device. The communication can be via microphone jack of the mobile device. Then, the recorded heart sound data can be uploaded from the mobile application into a backend cloud-based server. The classification scores can be improved with sharper spectrograms. Further, CNNs can be applied for accurate classification of heart sounds with intermediate spectrograms.

The CNN trained model can include neural network feature extractors that are trained from labelled examples to identify basic heart sounds, clicks and murmurs. In a preferred embodiment, the neural networks are of the time-delay variety, where the input span, number of layers, unit function, connectivity and initial weight selection are appropriately chosen according to well-known methods. However, it is to be appreciated that other types of neural networks can be used in accordance with the invention, while maintaining the spirit and scope thereof

The CNN trained model can implement neural networks for extraction of physiologically significant features from the audio slices or spectrograms (especially phonocardiograms); and these features can correspond to basic heart sounds, such as SI, or their components, such as Ml, Tl, murmurs, and so forth.

In an exemplary embodiment, the control unit 106 can execute one or more software units to analyse data from the patient. One unit monitors the patient's vital signs such as ECG/EKG and generates warnings. In this unit, vital signs can be collected and communicated to the database or database server 114 using wired or wireless transmitters. In one embodiment, the database server 114 feeds the data to a statistical analyser such as a neural network, which has been trained to flag potentially dangerous conditions. The neural network can be a back-propagation neural network, for example. In this embodiment, the statistical analyser can be trained with training data where certain signals are determined to be undesirable for the patient, given his age, weight, and physical limitations, among others. For example, the patient's glucose level should be within a well-established range, and any value outside of this range is flagged by the statistical analyser as a dangerous condition. As used herein, the dangerous condition can be specified as an event or a pattern that can cause physiological or psychological damage to the patient. Moreover, interactions between different vital signals can be accounted for so that the statistical analyser can take into consideration instances where individually the vital signs are acceptable, but in certain combinations, the vital signs can indicate potentially dangerous conditions. Once trained, the data received by the database server 114 can be appropriately scaled and processed by the statistical analyser. In addition to statistical analysers, the database server 114 can process vital signs using rule-based inference engines, fuzzy logic, as well as conventional if-then logic. Additionally, the database server 114 can process vital signs using Hidden Markov Models (HMMs), dynamic time warping, or template matching, among other.

In one embodiment, clustering operations are performed to detect patterns in the data. In another embodiment, a neural network is used to recognize each pattern as the neural network is quite robust at recognizing user habits or patterns. Once the treatment features have been characterized, the neural network then compares the input user information with stored templates of treatment vocabulary known by the neural network recognizer, among others. The recognition models can include a Hidden Markov Model (HMM), a dynamic programming model, a neural network, a fuzzy logic, or a template matcher, among others. These models can be used singly or in combination.

In one embodiment, feed forward artificial neural networks (NNs) can be used to classify valve-related heart disorders. The heart sounds are captured using the microphone or piezoelectric transducer. Relevant features can be extracted using several signal-processing tools, discrete wavelet transfer, fast Fourier transform, and linear prediction coding. The heart beat sounds are processed to extract the necessary features by: a) de-noising using wavelet analysis, b) separating one beat out of each record c) identifying each of the first heart sound (FHS) and the second heart sound (SHS). Valve problems are classified according to the time separation between the FHS and the SHS relative to cardiac cycle time, namely whether it is greater or smaller than 20% of cardiac cycle time. In one embodiment, the NN can include six nodes at both ends, with one hidden layer containing 10 nodes. In another embodiment, linear predictive code (LPC) coefficients for each event were fed to two separate neural networks containing hidden neurons.

In another embodiment, a normalized energy spectrum of the heart sound data is obtained by applying a Fast Fourier Transform. The various spectral resolutions and frequency ranges can be used as inputs into the NN to optimize these parameters to obtain the most favourable results.

In another embodiment, the heartbeats are de-noised using six-stage wavelet decomposition, thresholding, and then reconstruction. The feature extraction techniques to be used are the Decimation method, and the wavelet method. Classification of the heart diseases is done using Hidden Markov Models (HMMs) as well.

In yet another embodiment, a wavelet transform can be applied to a window of two periods of heart sounds. Two analysis realized for the signals in the window are segmentation of first and second heart sounds, and the extraction of the features. After segmentation, feature vectors are formed by using the wavelet detail coefficients at the sixth decomposition level. The best feature elements are analysed by using dynamic programming. In another embodiment, the wavelet decomposition and reconstruction method extract features from the heart sound recordings. An artificial neural network classification method classifies the heart sound signals into physiological and pathological murmurs. The heart sounds are segmented into four parts: the first heart sound, the systolic period, the second heart sound, and the diastolic period. The following features can be extracted and used in the classification algorithm: a) Peak intensity, peak timing, and the duration of the first heart sound b) the duration of the second heart sound c) peak intensity of the aortic component of S2(A2) and the pulmonic component of S2 (P2), the splitting interval and the reverse flag of A2 and P2, and the timing of A2 d) the duration, the three largest frequency components of the systolic signal and the shape of the envelope of systolic murmur e) the duration the three largest frequency components of the diastolic signal and the shape of the envelope of the diastolic murmur.

In an exemplary embodiment, a local signal analysis can be used with a classifier to detect, characterize, and interpret sounds corresponding to symptoms important for cardiac diagnosis. The system 100 detects a plurality of different heart conditions. Heart sounds are automatically segmented into a segment of a single heart beat cycle. Each segment can then be transformed using seven level wavelet decomposition, based on Coifman 4th order wavelet kernel. The resulting vectors with 4096 values, are reduced to 256 element feature vectors, this simplified the neural network and reduced noise.

In an exemplary embodiment, the classifier can be a CNN classifier. The CNN classifier is a standalone application running on cloud that can receive audio uploaded by user and classify it using the trained CNN model. The classifier and trainer can be python applications that can be implemented using Google's open source machine learning library TensorFlow.

In another embodiment, feature vectors can be formed by using wavelet detail and approximation coefficients at second and sixth decomposition levels. The classification (decision-making) is performed in four steps: segmentation of the first and second heart sounds, normalization process, feature extraction, and classification by the artificial neural network.

In an embodiment, the control unit 106 can be configured to compute any or a combination of a mean and standard deviation of the classification scores to remove any deviation (anomaly etc.), if present, in the classification scores. The control unit can be configured to store an audio slice corresponding to an obtained higher classification score in any or a combination of database 114 or in a CNN training database. The CNN training database can serve as a growing training database for re-training the CNN model for improved accuracy. Further, the heart signal classification along with scores can be transferred to mobile application installed on remote computing or mobile device.

In an embodiment, the CNN trained model can be configured to, based on any or a combination of the classification scores, the mean and the standard deviation of the classification scores, are retrained to enable detection of at least one of heart sound patterns and valvular heart disorders accurately and efficiently.

In an exemplary embodiment, neural networks can be trained on a training set that includes labels and corresponding data to classify objects from an input. For example, a first neural network can be trained on labeled images of various spectrograms and/or audio slices to identify different types of heart sounds. In some cases, it can be desirable to add new classes and/or modify the boundaries of existing classes after a network has been trained. Still, for various reasons, the training set can no longer be available after a first neural network has been trained. Therefore, because the training set is no longer available, incremental learning may not be performed on the network to add new classes and/or modify the boundaries of existing classes after a network has been trained. Therefore, it is desirable to transfer the learning of a first neural network to a second neural network to allow for incremental learning by the second neural network. For example, because the original training set may not be available after training the first neural network, the first neural network can be specified to label new data to train a second neural network that approximates the first neural network. The second neural network can then be used for incremental learning of other tasks.

Neural networks can be designed with a variety of connectivity patterns. In feed-forward networks, information is passed from lower to higher layers, with each neuron in a given layer communicating to neurons in higher layers. A hierarchical representation can be built up in successive layers of a feed-forward network, as described above. Neural networks can also have recurrent or feedback (also called top-down) connections. In a recurrent connection, the output from a neuron in a given layer is communicated to another neuron in the same layer. A recurrent architecture can be helpful in recognizing patterns that unfold in time. A connection from a neuron in a given layer to a neuron in a lower layer is called a feedback (or top-down) connection. A network with many feedback connections can be helpful when the recognition of a high-level concept can aid in discriminating the particular low-level features of an input. As mentioned above, the transfer learning can be implemented to retrain the CNN to enable accurate classification of spectrogram images as either normal or abnormal. The transfer learning can be commonly implemented to make small adjustments or modifications in the deep CNN model to enhance performance of the CNN model by replacing final layers of the model using a small set of training images or spectrograms.

In an exemplary embodiment, a deep CNN (DCN) can be trained with supervised learning. During training, a DCN can be presented with images or spectrograms of normal and abnormal heart sounds (and/or any other body sounds as well), such as converted spectrograms of the recorded heart sounds can then be computed to produce an output as normal heart sound or an abnormal heart sound. The output can be a vector of values corresponding to features such as health conditions corresponding to heart sounds. The network designer can want the DCN to output a high score for some of the neurons in the output feature vector, for example the ones corresponding to murmurs, arrhythmia as shown in the output for the CNN that has been trained. Before training, the output produced by the DCN is likely to be incorrect, and so an error can be calculated between the actual output and the target output. The weights of the DCN can then be adjusted so that the output scores of the DCN are more closely aligned with the target.

In an embodiment, the valvular heart disorders can include at least one of a murmur and an arrhythmia, and wherein the murmur can be at least one of a systolic murmur, late systolic murmur, holosystolic murmur and early diastolic murmur. Thus, patients can be saved by detecting heart disorders by the system 100.

In an exemplary embodiment, the system 100 can be implemented for early detection of any other body parts (such as respiratory organs such as lungs etc., kidneys, intestine etc.) that can rely on auscultation as a primary diagnosis technique.

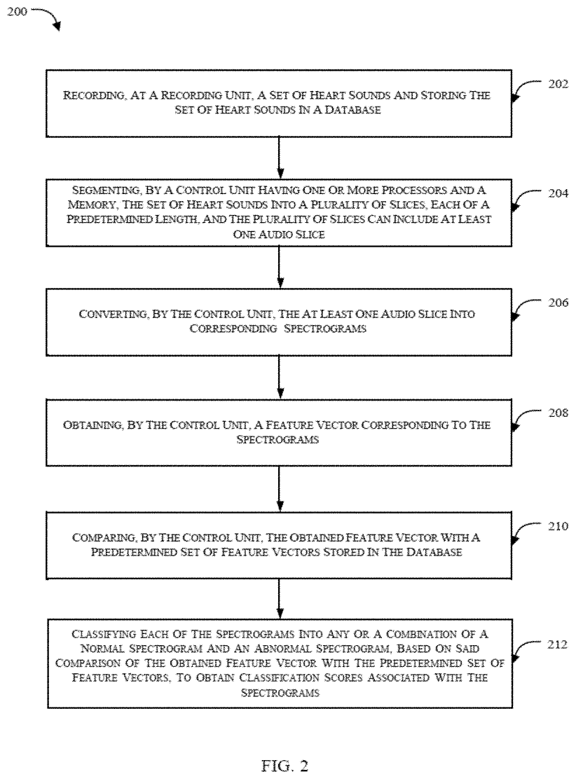

FIG. 2 illustrates an exemplary flow diagram representation of method for early detection of valvular heart disorders in a patient, in accordance with an embodiment of the present disclosure.

In an embodiment, the method 200 can include at a step 202, recording, at a recording unit, a set of heart sounds and storing the set of heart sounds in a database.

In an embodiment, the method 200 can include at a step 204, segmenting, by a control unit having one or more processors and a memory, the set of heart sounds into a plurality of slices, each of a predetermined length, and the plurality of slices can include at least one audio slice.

In an embodiment, the method 200 can include at a step 206, converting, by the control unit, the at least one audio slice into corresponding spectrograms.

In an embodiment, the method 200 can include at a step 208, obtaining, by the control unit, a feature vector corresponding to the spectrograms.

In an embodiment, the method 200 can include at a step 210, comparing, by the control unit, the obtained feature vector with a predetermined set of feature vectors stored in the database.

In an embodiment, the method 200 can include at a step 212, classifying each of the spectrograms into any or a combination of a normal spectrogram and an abnormal spectrogram, based on said comparison of the obtained feature vector with the predetermined set of feature vectors, to obtain classification scores associated with the spectrograms.

In an embodiment, at the step 212 of classifying each of the spectrograms, the control unit can be configured to classify, using a deep convolutional neural network (CNN) trained model, each of the spectrograms into any or a combination of the normal spectrogram and the abnormal spectrogram.

In an embodiment, the method 200 can include steps of: computing, at the one or more processors, any or a combination of a mean and standard deviation of the classification scores to remove any deviation, if present, in the classification scores; and storing, in the database, an audio slice corresponding to an obtained higher classification score.

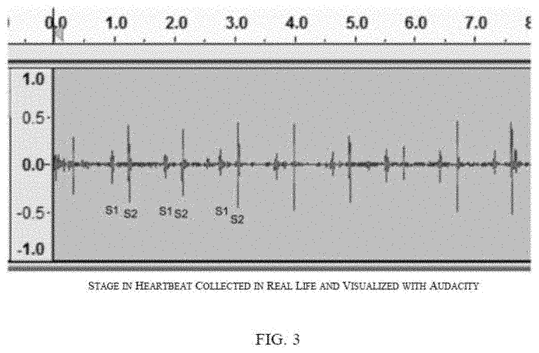

FIG. 3 illustrates an exemplary representation of heartbeat collected in real-life and visualized with audacity, in accordance with an embodiment of the present disclosure. The cardiac cycles follow a pattern with each normal heartbeat having a sequence of two heart sounds (S1 & S2).

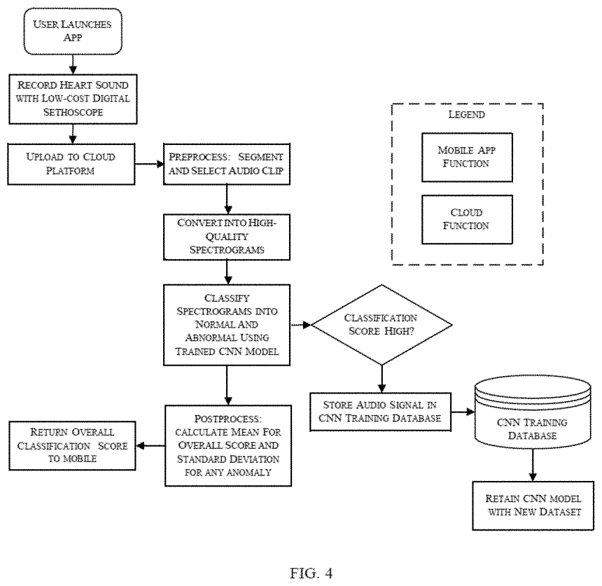

FIG. 4 illustrates an exemplary flow diagram representation of an algorithm for automated screening, in accordance with an embodiment of the present disclosure. As shown in FIG. 4, after launching or installing application interface in any computing device (mobile device, laptop etc.), heart sounds can be recorded with a low-cost digital stethoscope. Then heart sounds can be pre-processed i.e. they are segmented and audio clips can be selected and converted into high quality spectrograms. The spectrograms can be classified into normal and abnormal by using trained CNN model. Then during post-processing, mean can be calculated for overall classification and standard deviation can be calculated for any anomaly. Further, if classification score is higher, then an audio signal corresponding to the higher classification score can be stored in CNN training database that can enable retraining of CNN model with new dataset.

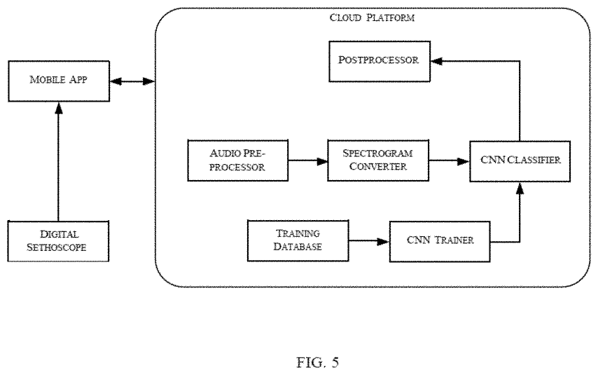

FIG. 5 illustrates an exemplary end-to-end system architecture for diagnostic tool, in accordance with an embodiment of the present disclosure. As shown in FIG. 5, mobile application can be designed using any software and can be installed in a mobile device. An audio pre-processor and a spectrum converter can be configured to slice audio files of audio sounds and plot spectrograms with libraries such as scipy and matplotlib. The program performs a short time Fourier transform (STFT) of the audio signal. A CNN trainer can be an asynchronous application configured to train CNN model with training dataset of spectrograms generated from heart sound database. A CNN classifier can be configured to receive audio uploaded by user and classify it using the trained CNN model. A post processor can be a cloud program that can calculate mean of classification scores of spectrograms of all slices of audio signal as the overall classification score, and can compute standard deviation to detect any anomaly. A cloud database stores user-preloaded audio signals along with metadata for retraining the CNN model.

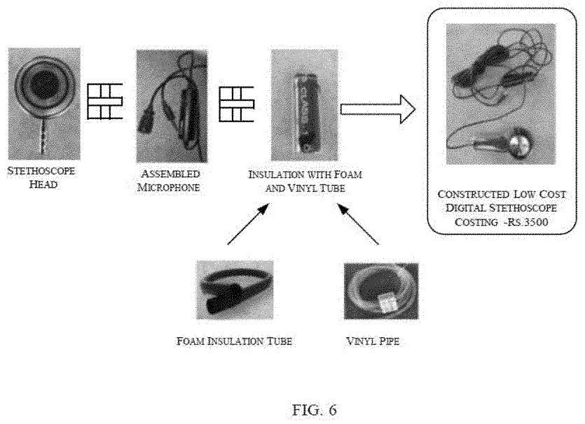

FIG. 6 illustrates an exemplary representation of digital stethoscope, in accordance with an embodiment of the present disclosure. It can be built using an analog stethoscope head, an 8 mm lapel mike (Omni-directional electrical condenser with signal-to-noise ratio of 74 dB, sensitivity of −30 dB, frequency range of 65 Hz-18 KHz), and a foam insulator with vinyl tube to provide a sound insulated connection. To help record low amplitude and lower frequency band heart sound, the sensitivity can be maximized; a low pass filter can be used, and 32-bit floating-point sampling at rate of 44 KHz can be used for recording. Audacity can be used to tune the audio fidelity and Sonic Visualizer for experimental view of spectrograms on a laptop or any other computing device.

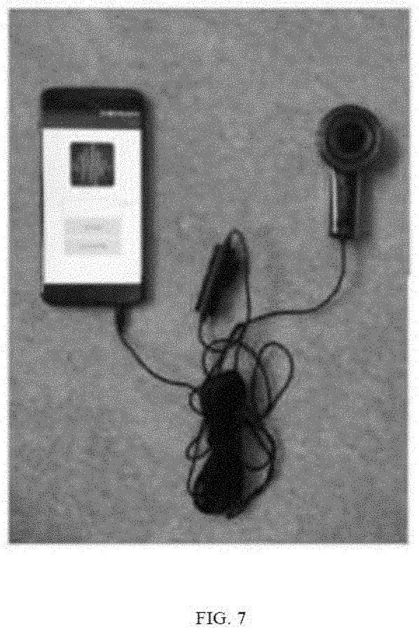

FIG. 7 illustrates an exemplary representation of mobile device in connection with digital stethoscope, in accordance with an embodiment of the present disclosure.

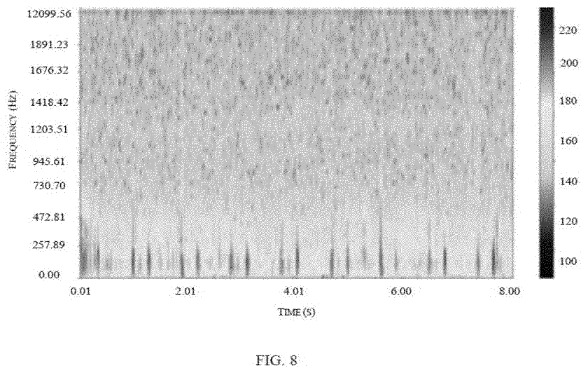

FIG. 8 illustrates an exemplary representation of spectrogram of real heart sample collected with stethoscope, in accordance with an embodiment of the present disclosure. Spectrograms are 2D or 3D images representing sequences of spectra with time along one axis, frequency along the other, and brightness or color representing the strength of a frequency component at each time frame. Therefore, spectrograms can be considered a very detailed image of the audio signal shown on a graph according to time and frequency with brightness or height (3D) representing amplitude. Spectrograms can be created either using a Fourier Transform of the time based signal or approximated with a series of band-pass filter banks. With digitally sampled audio data, the Fast Fourier Transform (FFT) method can be implemented. A spectral image can include slices of overlapping images (“windowing”) with each slice representing the frequency components and strength at the time. This method is called Short-Time-Fourier-Transform (STFT). The size and shape of the windowing slices can be varied to provide tuneable parameters for our spectrogram image. The trade-off parameters are window length, window type, FFT length and hop size. Use of shorter time window results in better timing precision at the expense of frequency precision and vice versa. The window types (Rectangular, Gaussian, Hamming, Hanning, Kaiser etc.) controls side-lobe suppression and the FFT length determines the amount of spectral oversampling. A Hanning window can be used and can experiment with other trade off parameters in CNN model model for optimal classification results.

Once the audio signal is translated into an image, CNNs are naturally the best candidate to identify patterns within the image and classify the spectrogram. Given the natural rhythm and repetitive pattern of the cardiac cycle and a persistent signature of the abnormality (murmur and arrhythmia) within few systolic beats, time slices of the audio signal can be taken to generate the spectrogram.

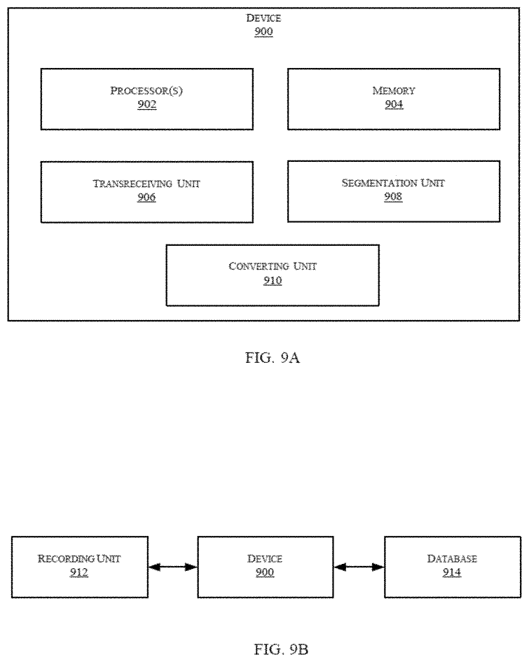

FIG. 9A illustrates an exemplary block diagram representation of device for early detection of valvular heart disorders in a patient, in accordance with an embodiment of the present disclosure.

FIG. 9B illustrates an exemplary schematic representation of the device of FIG. 9A with recording unit and database, in accordance with an embodiment of the present disclosure.

The device 900 can include one or more processors 902 and a memory 904 that can be operatively coupled with the processors 902.

The memory 904 can store computer implemented instructions which when executed by the processors 902 to enable the device 900 to: receive, using a transceiving unit 906, a recorded set of heart sounds from a recording unit 912 that can be operatively coupled to the device 900; and segment, using a segmentation unit 908, the received set of heart sounds into a plurality of slices, each of a predetermined length, and the plurality of slices can include at least one audio slice.

The device 900 can be configured to: convert, using a converting unit 910, the at least one audio slice into corresponding spectrograms; and obtain, at the processors 902, a feature vector corresponding to the spectrograms.

The device 900 can be configured to compare, at processors 902, the obtained feature vector with a predetermined set of feature vectors that can be stored in a database 914 operatively coupled to the device 900.

The device 900 can be configured to classify, at the processors 902, each of the spectrograms into any or a combination of a normal spectrogram and an abnormal spectrogram, based on said comparison of the obtained feature vector with the predetermined set of feature vectors, to obtain classification scores associated with the spectrograms. The spectrograms can be time-based spectrograms

In an embodiment, the device 900 can be configured to classify, using a deep convolutional neural network (CNN) trained model, each of the spectrograms into any or a combination of the normal spectrogram and the abnormal spectrogram.

In an exemplary embodiment, the classification can be automated by using any or a combination of artificial neural network-based techniques, support vector machines, hidden Markov model-based and clustering-based approaches. Further, implementation of Convolutional Neural Networks (CNN) and Residual Neural Networks (RNN) can be done to achieve better accuracy results.

In an embodiment, the device 900 can be configured to: compute any or a combination of a mean and standard deviation of the classification scores to remove any deviation, if present, in the classification scores; and store, in the database 914, an audio slice corresponding to an obtained higher classification score.

In an embodiment, the CNN trained model that can be implemented in the device 900 can be configured to, based on any or a combination of the classification scores, the mean and the standard deviation of the classification scores, detect at least one of heart sound patterns and valvular heart disorders. The device 900 can be a portable device.

In an exemplary embodiment, abnormal category can cover a range of murmurs such as early systolic murmur, late systolic murmur, holosystolic murmur, early diastolic murmur etc. Each audio file can split into 8 second slices, which can be fed into the spectrogram generator configured with the following parameters such as overlap factor=0.9, frequency binsize=2**12, scale factor=2 etc.

In an exemplary embodiment, the CNN trainer can be configured with 4000 training steps (transfer learning) with spectrograms from normal and abnormal categories. Trained model can be used to classify test spectrograms. The accuracy of classification can be captured as a probability score that represents the confidence measure in the output of classification. Results show that the classifier can be recognized as normal and abnormal heart sounds correctly based on the spectrogram training.

In an exemplary embodiment, reducing the spectrogram overlap factor from 0.9 to 0.5, reducing the frequency binsize from 2**12 to 2**10 and increasing the scale from 2 to 20, can led to favourable results. By changing the time vs frequency granularity, the parameters that work optimally with the CNN can be able to better discern between the normal and abnormal categories. Training and testing can rerun with the set of new spectrograms generated with the new parameters. The result demonstrates a higher classification accuracy with sharper spectrograms that can have better distinguishable features between normal and abnormal categories. The smoother spectrogram with optimal parameters can result in higher accuracy.

In an exemplary embodiment, the system 100, method 200 and device 900 can be helpful for unskilled health workers in detecting early signs of valvular heart disorders, and can help in simulating cardiac auscultation expertise of a trained professional in order to bridge the lack of expertise of some untrained professionals. This can also ensure high quality in training and testing data. This can be helpful in referring patient to advanced tests like ECG etc.

While the foregoing describes various embodiments of the invention, other and further embodiments of the invention can be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### ADVANTAGES OF THE PRESENT DISCLOSURE

The present disclosure provides a system, device and method for early detection or screening of heart related disorders or abnormalities such as murmurs etc. in a patient.

The present disclosure provides a simple and cost effective system, device and method for early detection of valvular heart disorders in a patient.

The present disclosure provides a reliable, efficient and accurate system, device and method for early detection of valvular heart disorders in a patient.

The present disclosure provides an automated stethoscope based cardiac auscultation to detect abnormal heart sound to save patients from heart related diseases.

We claim:

1. A system for detection of valvular heart disorders, the system comprising:
a recording unit configured to record a set of heart sounds and store the set of heart sounds in a database; and
a control unit comprising one or more processors and a memory operatively coupled with the one or more processors, the memory storing instructions executable by the one or more processors to enable the control unit to:
segment the set of heart sounds into a plurality of slices, each of a predetermined length, where each of the plurality of slices comprises at least one audio slice;
convert the at least one audio slice into corresponding one or more spectrograms;
obtain at least one feature vector corresponding to the one or more spectrograms;
compare the obtained at least one feature vector with a predetermined set of feature vectors stored in the database; and
classify each of the one or more spectrograms into any or a combination of a normal spectrogram and an abnormal spectrogram, based on said comparison of the obtained feature vector with the predetermined set of feature vectors, to obtain classification scores associated with the one or more spectrograms.

2. The system as claimed in claim 1, wherein the one or more spectrograms are time-based spectrograms.

3. The system as claimed in claim 1, wherein the control unit is configured to classify, using a deep convolutional neural network (CNN) trained model, each of the one or more spectrograms into any or a combination of the normal spectrogram and the abnormal spectrogram.

4. The system as claimed in claim 3, wherein the system is configured to:
compute any or a combination of a mean and standard deviation of the classification scores to remove any deviation, if present, in the classification scores; and
store, in the database, an audio slice corresponding to an obtained higher classification score.

5. The system as claimed in claim 4, wherein the CNN trained model is configured to, based on any or a combination of the classification scores, the mean and the standard deviation of the classification scores, detect at least one of heart sound patterns and valvular heart disorders.

6. The system as claimed in claim 1, wherein the valvular heart disorders comprises at least one of a murmur and an arrhythmia, and wherein the murmur is at least one of a systolic murmur, late systolic murmur, holosystolic murmur and early diastolic murmur.

7. A method for detection of valvular heart disorders, the method comprising steps of:
recording, at a recording unit, a set of heart sounds and storing the set of heart sounds in a database;
segmenting, by a control unit having one or more processors and a memory, the set of heart sounds into a plurality of slices, each of a predetermined length, the plurality of slices comprises at least one audio slice;
converting, by the control unit, the at least one audio slice into corresponding one or more spectrograms;
obtaining, by the control unit, a feature vector corresponding to the one or more spectrograms;
comparing, by the control unit, the obtained feature vector with a predetermined set of feature vectors stored in the database; and
classifying each of the one or more spectrograms into any or a combination of a normal spectrogram and an abnormal spectrogram, based on said comparison of the obtained feature vector with the predetermined set of feature vectors, to obtain classification scores associated with the one or more spectrograms.

8. The method as claimed in claim 7, wherein at the step of classifying each of the one or more spectrograms, the control unit is configured to classify, using a deep convolutional neural network (CNN) trained model, each of the one or more spectrograms into any or a combination of the normal spectrogram and the abnormal spectrogram.

9. The method as claimed in claim 7, wherein the method comprises steps of:
computing, at the one or more processors, any or a combination of a mean and standard deviation of the classification scores to remove any deviation, if present, in the classification scores; and
storing, in the database, an audio slice corresponding to an obtained higher classification score.

10. A device for detection of valvular heart disorders, the device comprising one or more processors and a memory operatively coupled with the one or more processors, the memory storing instructions executable by the one or more processors to enable the device to:
receive, using a transceiving unit, a recorded set of heart sounds from a recording unit operatively coupled to the device;
segment, using a segmentation unit, the received set of heart sounds into a plurality of slices, each of a predetermined length, the plurality of slices comprises at least one audio slice;
convert, using a converting unit, the at least one audio slice into corresponding one or more spectrograms;
obtain, at the one or more processors, a feature vector corresponding to the one or more spectrograms;
compare, at the one or more processors, the obtained feature vector with a predetermined set of feature vectors stored in a database operatively coupled to the device; and
classify, at the one or more processors, each of the one or more spectrograms into any or a combination of a normal spectrogram and an abnormal spectrogram, based on said comparison of the obtained feature vector with the predetermined set of feature vectors, to obtain classification scores associated with the one or more spectrograms.
